(19) United States
(12) Patent Application Publication
(10) Pub. No.: US 2005/0026853 A1
(43) Pub. Date: Feb. 3, 2005

(54) Anti-viral 7-deaza D-nucleosides and uses thereof

(75) Inventors: Khalid Mekouar, Laval (CA); Robert Deziel, Montreal (CA)

Correspondence Address: SEED INTELLECTUAL PROPERTY LAW GROUP PLLC, 701 FIFTH AVE, SUITE 6300, SEATTLE, WA, 98104-7092, US

(73) Assignee: Micrologix Biotech Inc., Vancouver (CA)

(21) Appl. No.: 10/627,586

(22) Filed: Jul. 25, 2003

Related U.S. Application Data
(60) Provisional application No. 60/398,424, filed on Jul. 25, 2002.

(51) Int. Cl. A61K 31/7076; C07H 19/22

(52) U.S. Cl. 514/43; 536/27.13

(57) ABSTRACT

The present invention relates generally to anti-viral compounds, particularly anti-viral 7-deaza D-nucleosides and analogues, or derivatives thereof. The invention also relates to the use of such compounds to treat or prevent hepatitis B virus (HBV) infections, and to the use of such compounds to examine the biological mechanisms of HBV infection.

### CROSS-REFERENCE TO RELATED APPLICATION

This application claims the benefit of U.S. Provisional Patent Application No. 60/398,424, filed Jul. 25, 2002, wherein this provisional application is incorporated herein by reference in its entirety.

### BACKGROUND OF THE INVENTION

1. Field of the Invention

The present invention relates generally to the treatment of infectious disease, and more specifically, to methods and compounds for the preparation and therapeutic use of anti-viral agents, particularly anti-viral D-nucleosides and derivatives thereof, and more particularly anti-viral 7-deaza D-nucleosides and derivatives thereof.

2. Description of Related Art

Viral infections, such as that caused by human hepatitis B virus (HBV), are a leading cause of liver disease, and can progress to more serious complications, such as cirrhosis and hepatocellular carcinoma (HCC). Nucleoside and nucleotide analogs have long been studied as potential antiviral compounds.

For example, a number of D-nucleoside analogs have been investigated and are presently used as anti-viral agents, including HIV reverse transcriptase inhibitors (such as AZT, ddI, ddC and d4T). Some nucleoside analogues, including 7-deazaguanosine and 3-deazaguanine nucleosides and nucleotides, have demonstrated anti-viral activity against certain DNA and RNA viruses (see, e.g., Revanker et. al. J. Med. Chem. 27:1389, 1984). Certain 7- and 9-deazaguanine C-nucleosides exhibit the ability to protect mice against lethal challenge of Semliki Forest Virus (Girgis et al., J. Med. Chem. 33:2750, 1990). Yet others have been examined as immunomodulators (see, e.g., Weigle, W. O., CRC Crit Rev. Immunol. 7:285, 1987 (for a review); Lin et al., J. Med. Chem. 28:1194, 1985; Reitz et al., J. Med. Chem. 27:3561, 1994 and Michael et al., J. Med. Chem. 36:3431, 1993; Bonnet et al., J. Med. Chem. 36:635, 1993; U.S. Pat. Nos. 4,328,336 and 5,041,542). Similarly, purine L-nucleoside analogs have been investigated as antiviral agents (see, e.g., WO 98/16184).

With regard to HBV infections, a number of strategies have been used in an attempt to treat chronic HBV infection. The most common treatment includes the use of lamivudine (3TC) and interferon-α, and recently approved in the U.S. is use of adefovir dipivoxil. These existing treatments, however, continue to produce unwanted side-effects, and, in some cases, show limited efficacy.

Thus, a need exists for identifying and developing anti-viral agents having improved activity and reduced toxicity (and do not cause other undesirable side effects), therapeutics for the treatment of HBV. The present invention meets such needs, and further provides other related advantages.

### SUMMARY OF THE INVENTION

The present invention generally provides nucleoside derivatives, in particular, 7-deaza D-nucleosides, and compositions of such compounds for use in treating or preventing, for example, viral infections such as those caused by hepatitis B virus (HBV). In particular, the present invention provides 7-deaza D-nucleosides and analogues, and derivatives thereof, having unexpectedly high inhibitory activity against HBV.

In one aspect, the invention provides anti-viral compounds according to structure (I): 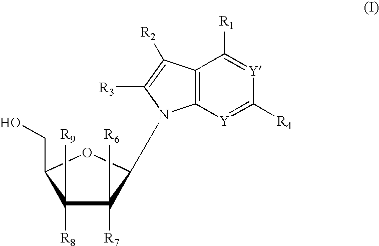
and pharmaceutically acceptable salts thereof wherein R¹ is hydrogen, C₁-C₆ alkyl, Cl, OH, C₁-C₄ alkoxy, NH₂, or NHZR⁵; each of R² and R³ are independently hydrogen, C₁-C₆ alkyl, methyl, C₂-C₆ alkenyl, C₂-C₆ alkynyl, Cl, I, Br, F, heterocyclyl, or R² and R³ together with the carbons to which they are attached form a 5-membered ring; R⁴ is hydrogen, OH, C₁-C₆ alkyl, C₁-C₆ alkenyl, C₁-C₄ alkoxy, NH₂, NHZR⁵ or N(R⁵)₂; each R⁵ is independently C₁-C₆ alkyl, C₅-C₆ alkenyl, cycloalkyl, or aryl; each of R⁶, R⁷, R⁸ and R⁹ are independently hydrogen, OH, C₁-C₆ alkyl, NH₂, NHZR⁵, F, Cl, or Br, or R⁶, R⁷, R⁸ and R⁹ form an epoxide or a double bond; each of Y and Y′ are independently N or CH; and Z is CO, C(O)NH or SO₂. In certain embodiments, any of the aforementioned compounds wherein R¹ is NH₂, R² is a halogen or C₁-C₄ alkyl, and R³ and R⁴ are hydrogen; or R¹ is NH₂, R² is hydrogen or a halogen; R³ is a halogen or C₁-C₄ alkyl, and R⁴ is hydrogen; or R¹ is NH₂, each of R² and R³ are independently hydrogen or a halogen; and R⁴ is C₁-C₄ alkyl; or R¹ is NH₂, R² and R³ together with the carbon atoms they are attached to form a pentene ring, and R⁴ is hydrogen; or R¹ is hydrogen or C₁-C₄ alkyl, each of R² and R³ are independently hydrogen or a halogen; and R⁴ is hydrogen; or R¹ is NH2, each of R² and R³ are independently hydrogen or a halogen; and R⁴ is —NHZR⁵. In still other embodiments, any of the aforementioned compounds wherein R⁶, R⁷, R⁸ and R⁹ are hydrogen; or R⁶, R⁸ and R⁹ are hydrogen, and R⁷ is OH; or R⁶ and R⁹ are hydrogen, R⁷ is C₁-C₄ alkyl, and R⁸ is OH; or R⁶ and R⁹ are hydrogen, R⁷ is NHZR⁵, and R⁸ is OH; or R⁶ and R⁹ are hydrogen, R⁷ is F, and R⁸ is OH; or R⁶ is C₁-C₄ alkyl, R⁷ and R⁹ are hydrogen, and R⁸is OH. In another embodiment, the compound has structure (II):

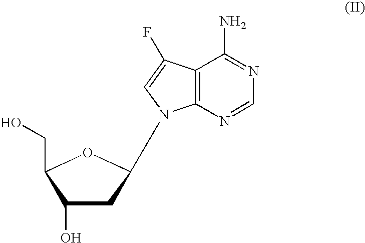

In another aspect, the invention provides a pharmaceutical composition comprising any of the aforementioned compounds and a pharmaceutically acceptable carrier, excipient or diluent. In other embodiments, the composition further comprises an adjuvant, such as alum. In another embodiment, the composition further comprises other antimicrobial agents, such as one or more antibiotic, antifungal, anti-inflammatory, and other anti-viral.

In still another aspect, the invention provides a method of treating or preventing a viral infection, comprising administering to a subject in need thereof any of the aforementioned anti-viral compounds or a composition of such compounds in an amount effective to treat or prevent a viral infection. In certain embodiments, the anti-viral compound or composition is administered orally, topically, or systemically. In another preferred embodiment, the composition is administered to treat or prevent a viral infection caused by hepatitis B virus (HBV).

### BRIEF DESCRIPTION OF THE DRAWINGS

FIG. 1 shows a reaction scheme for generating intermediate compound 4-chloro-5-fluoro-7H-pyrrolo[2, 3-d] pyrimidine 3.

FIG. 2 shows a reaction scheme for generating anti-viral compound 4-amino-5-fluoro-7-(2′-deoxy-β-D-erythro-pentofuranosyl) pyrrolo[2, 3-d] pyrimidine 6.

### DETAILED DESCRIPTION OF THE INVENTION

As set forth above, the present invention provides compositions and methods for using and making anti-viral nucleoside analogues, and derivatives thereof, to treat or prevent infectious diseases. In particular, these nucleoside analogues, and derivatives thereof, are useful for treating or preventing viral infections, such as hepatitis B virus (HBV) infections. The invention, therefore, relates generally to the surprising discovery that certain nucleoside analogues, and derivatives thereof, have an unexpectedly high activity against HBV. Accordingly, the compounds of the invention are useful, for example, as research tools for in vitro and cell-based assays to study the biological mechanisms of HBV infection (e.g., replication and transmission), and are useful as potential therapeutics for the prevention or treatment of HBV infection and HBV related disease. Discussed in more detail below are nucleoside analogues, and derivatives thereof, suitable for use within the present invention, as well as representative compositions and therapeutic uses.

Prior to setting forth the invention in more detail, it may be helpful to an understanding thereof to set forth definitions of certain terms to be used hereinafter.

In the present description, any concentration range, percentage range, or integer range is to be understood to include the value of any integer within the recited range and, when appropriate, fractions thereof (such as one tenth and one hundredth of an integer), unless otherwise indicated. As used herein, “about” or “comprising essentially of” mean±15%. The use of the alternative (e.g., “or”) should be understood to mean either one, both or any combination thereof of the alternatives. In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present invention.

As used herein, the term “alkyl” refers to a saturated or unsaturated, branched, straight-chain or cyclic monovalent hydrocarbon group derived by the removal of one hydrogen atom from a single carbon atom of a parent alkane, alkene or alkyne. Typical alkyl groups include methyl; ethyls such as ethanyl, ethenyl, ethynyl; propyls such as propan-1-yl, propan-2-yl, cyclopropan-1-yl, prop-1-en-1-yl, prop-1-en-2-yl, prop-2-en-1-yl (allyl), cycloprop-1-en-1-yl; cycloprop-2-en-1-yl, prop-1-yn-1-yl , prop-2-yn-1-yl, etc.; butyls such as butan-1-yl, butan-2-yl, 2-methyl-propan-1-yl, 2-methyl-propan-2-yl, cyclobutan-1-yl, but-1-en-1-yl, but-1-en-2-yl, 2-methyl-prop-1-en-1-yl, but-2-en-1-yl , but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, but-1-yn-1-yl, but-1-yn-3-yl, but-3-yn-1-yl, etc.; and the like.

The term “alkyl” is specifically intended to include straight- or branched-hydrocarbons having from 1 to 25 carbon atoms, more preferably 5 to 20, and most preferably 10 to 18. The alkyls may have any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. When a specific level of saturation is intended, the expressions “alkanyl,” “alkenyl,” and “alkynyl” are used. The expression “lower alkyl” refers to alkyl groups comprising from 1 to 8 carbon atoms. The alkyl group may be substituted or unsubstituted.

“Alkanyl” refers to a saturated branched, straight-chain or cyclic alkyl group. Typical alkanyl groups include methanyl; ethanyl; propanyls such as propan-1-yl, propan-2-yl (isopropyl), cyclopropan-1-yl, etc.; butyanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkenyl” refers to an unsaturated branched, straight-chain, cyclic alkyl group, or combinations thereof having at least one carbon-carbon double bond derived by the removal of one hydrogen atom from a single carbon atom of a parent alkene. The group may be in either the cis or trans conformation about the double bond(s). Typical alkenyl groups include ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl , but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like. The alkenyl group may be substituted or unsubstituted.

“Alkynyl” refers to an unsaturated branched, straight chain or cyclic alkyl group having at least one carbon-carbon triple bond derived by the removal of one hydrogen atom from a single carbon atom of a parent alkyne. Typical alkynyl groups include ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Alkyldiyl” refers to a saturated or unsaturated, branched, straight-chain or cyclic divalent hydrocarbon group derived by the removal of one hydrogen atom from each of two different carbon atoms of a parent alkane, alkene or alkyne, or by the removal of two hydrogen atoms from a single carbon atom of a parent alkane, alkene or alkyne. The two monovalent radical centers or each valency of the divalent radical center can form bonds with the same or different atoms. Typical alkyldiyl groups include methandiyl; ethyldiyls such as ethan-1,1-diyl, ethan-1,2-diyl, ethen-1,1-diyl, ethen-1,2-diyl; propyldiyls such as propan-1,1-diyl, propan-1,2-diyl, propan-2,2-diyl, propan-1,3-diyl, cyclopropan-1,1-diyl, cyclopropan-1,2-diyl, prop-1-en-1,1-diyl, prop-1-en-1,2-diyl, prop-2-en-1,2-diyl, prop-1-en-1,3-diyl, cycloprop-1-en-1,2-diyl, cycloprop-2-en-1,2-diyl, cycloprop-2-en-1,1-diyl, prop-1-yn-1,3-diyl, etc.; butyidiyls such as, butan-1,1-diyl, butan-1,2-diyl, butan-1,3-diyl, butan-1,4-diyl, butan-2,2-diyl, 2-methyl-propan-1,1-diyl, 2-methyl-propan-1,2-diyl, cyclobutan-1,1-diyl; cyclobutan-1,2-diyl, cyclobutan-1,3-diyl, but-1-en-1,1-diyl, but-1-en-1,2-diyl, but-1-en-1,3-diyl, but-1-en-1,4-diyl, 2-methyl-prop-1-en-1,1-diyl, 2-methanylidene-propan-1,1-diyl, buta-1,3-dien-1,1-diyl, buta-1,3-dien-1,2-diyl, buta-1,3-dien-1,3-diyl, buta-1,3-dien-1,4-diyl, cyclobut-1-en-1,2-diyl, cyclobut-1-en-1,3-diyl, cyclobut-2-en-1,2-diyl, cyclobuta-1,3-dien-1,2-diyl, cyclobuta-1,3-dien-1,3-diyl, but-1-yn-1,3-diyl, but-1-yn-1,4-diyl, buta-1,3-diyn-1,4-diyl, etc.; and the like. When a specific level of saturation is intended, the nomenclature alkanyidiyl, alkenyldiyl or alkynyldiyl is used. In preferred embodiments, the alkyldiyl group is (C₁-C₄) alkyldiyl. Also preferred are saturated acyclic alkanyldiyl groups in which the radical centers are at the terminal carbons, e.g., methandiyl (methano); ethan-1,2-diyl (ethano); propan-1,3-diyl (propano); butan-1,4-diyl (butano); and the like (also referred to as alkylenos, defined infra).

“Alkyleno” refers to a straight-chain alkyldiyl group having two terminal monovalent radical centers derived by the removal of one hydrogen atom from each of the two terminal carbon atoms of straight-chain parent alkane, alkene or alkyne. Typical alkyleno groups include methano; ethylenos such as ethano, etheno, ethyno; propylenos such as propano, prop[1]eno, propa[1,2]dieno, prop[1]yno, etc.; butylenos such as butano, but[1]eno, but[2]eno, buta[1,3]dieno, but[1]yno, but[2]yno, but[1,3]diyno, etc.; and the like. When a specific level of saturation is intended, the nomenclature alkano, alkeno or alkyno is used. In preferred embodiments, the alkyleno group is (C₁-C₆) or (C₁-C₄) alkyleno. Also preferred are straight-chain saturated alkano groups, e.g., methano, ethano, propano, butano, and the like.

“Heteroalkyl, Heteroalkanyl, Heteroalkenyl, Heteroalkanyl, Heteroalkyldiyl and Heteroalkyleno” refer to alkyl, alkanyl, alkenyl, alkynyl, alkyldiyl and alkyleno groups, respectively, in which one or more of the carbon atoms (and any associated hydrogen atoms) are each independently replaced with the same or different heteroatoms or heteroatomic groups. Typical heteroatoms or heteroatomic groups that can be included in these groups include —O—, —S—, —Se—, —O—O—, —S—S—, —O—S—, —O—S—O—, —O—NR′—, —NR′—, —NR′—NR′—, ═N—N═, —N═N—, —N═N—NR′—, —PH—, —P(O)₂—, —O—P(O)₂—, —SH₂—, —S(O)₂—, —SnH₂— and the like, and combinations thereof, including —NR′—S(O)₂—, where each R′ is independently selected from hydrogen, alkyl, alkanyl, alkenyl, alkynyl, aryl, arylalkyl, heteroaryl and heteroarylalkyl, as defined herein.

“Aryl” refers to a monovalent aromatic hydrocarbon group derived by the removal of one hydrogen atom from a single carbon atom of a parent aromatic ring system. Typical aryl groups include groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In preferred embodiments, the aryl group is (C₅-C₁₄) aryl, with (C₅-C₁₀) being even more preferred. Particularly preferred aryls are cyclopentadienyl, phenyl and naphthyl. The aryl group may be substituted or unsubstituted.

“Arylalkyl” refers to an acyclic alkyl group in which one of the hydrogen atoms bonded to a carbon atom, typically a terminal or sp³ carbon atom, is replaced with an aryl group. Typical arylalkyl groups include benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylakenyl or arylalkynyl is used. In preferred embodiments, the arylalkyl group is (C₆-C₂₀) arylalkyl, e.g., the alkanyl, alkenyl or alkynyl moiety of the arylalkyl group is (C₁-C₆) and the aryl moiety is (C₅-C₁₄). In particularly preferred embodiments the arylalkyl group is (C₆-C₁₃), e.g., the alkanyl, alkenyl or alkynyl moiety of the arylalkyl group is (C₁-C₃) and the aryl moiety is (C₅-C₁₀).

“Heteroaryl” refers to a monovalent heteroaromatic group derived by the removal of one hydrogen atom from a single atom of a parent heteroaromatic ring system, which may be monocyclic or fused ring (i.e., rings that share an adjacent pair of atoms). Typical heteroaryl groups include groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In preferred embodiments, the heteroaryl group is a 5-14 membered heteroaryl, with 5-10 membered heteroaryl being particularly preferred. The most preferred heteroaryl groups are those derived from thiophene, pyrrole, benzothiophene, benzofuran, indole, pyridine, quinoline, imidazole, oxazole and pyrazine. The heteroaryl group may be substituted or unsubstituted.

“Heteroalicyclic” refers to a monocyclic or fused ring group having in the ring(s) one or more atoms selected preferably from nitrogen, oxygen and sulfur. The rings may also have one or more double bonds. However, the rings do not have a completely conjugated π-electron system. The heteroalicyclic ring may be substituted or unsubstituted. When substituted, the substituted group(s) preferably are selected independently from alkyl, aryl, haloalkyl, halo, hydroxy, alkoxy, mercapto, cyano, sulfonamidyl, aminosulfonyl, acyl, acyloxy, vitro, and substituted amino.

“Heteroarylalkyl” refers to an acyclic alkyl group in which one of the hydrogen atoms bonded to a carbon atom, typically a terminal or sp³ carbon atom, is replaced with a heteroaryl group. When one or more specific alkyl moiety is intended, the nomenclature heteroarylalkanyl, heteroarylakenyl or heterorylalkynyl is used. In preferred embodiments, the heteroarylalkyl group is a 6-20 membered heteroarylalkyl, e.g., the alkanyl, alkenyl or alkynyl moiety of the heteroarylalkyl is 1-6 membered and the heteroaryl moiety is a 5-14-membered heteroaryl. In particularly preferred embodiments, the heteroarylalkyl is a 6-13 membered heteroarylalkyl, e.g., the alkanyl, alkenyl or alkynyl moiety is 1-3 membered and the heteroaryl moiety is a 5-10 membered heteroaryl.

“Halogen” or “halo” refers to fluoro (F), chloro (Cl), bromo (Br), iodo (I). As used herein, —X refers to independently any halogen.

“Acyl” group refers to the C(O)—R″ group, where R″ is selected preferably from hydrogen, hydroxy, alkyl, haloalkyl, cycloalkyl, aryl optionally substituted with one or more alkyl, haloalkyl, alkoxy, halo and substituted amino groups, heteroaryl (bonded through a ring carbon) optionally substituted with one or more alkyl, haloalkyl, alkoxy, halo and substituted amino groups and heteroalicyclic (bonded through a ring carbon) optionally substituted with one or more alkyl, haloalkyl, alkoxy, halo and substituted amino groups. Acyl groups include aldehydes, ketones, acids, acid halides, esters and amides. Preferred acyl groups are carboxy groups, e.g., acids and esters. Esters include amino acid ester derivatives. The acyl group may be attached to a compound's backbone at either end of the acyl group, i.e., via the C or the R″. When the acyl group is attached via the R″, then C will bear another substituent, such as hydrogen, alkyl, and the like.

“Substituted” refers to a group in which one or more hydrogen atoms are each independently replaced with the same or different substituent(s). Typical substituents include —X, —R¹³, —O—, ═O, —OR, —SR¹³, —S—, ═S, —NR¹³R¹³, ═NR¹³, CX₃, —CF₃, —CN, —OCN, —SCN, —NO, —NO₂, ═N₂, —N₃, —S(O)₂O—, —S(O)₂OH, —S(O)₂R¹³, —OS(O₂)O—, —OS(O)₂OH, —OS(O)₂R¹³, —P(O)(O⁻)₂, —P(O)(OH)(O⁻), —OP(O)₂(O⁻), —C(O)R¹³, —C(S)R¹³, —C(O)OR¹³, —C(O)O⁻, —C(S)OR¹³, and —C(NR¹³)NR¹³R¹³, wherein each X is independently a halogen; each R¹³ is independently hydrogen, halogen, alkyl, aryl, arylalkyl, arylaryl, arylheteroalkyl, heteroaryl, heteroarylalkyl NR¹⁴R¹⁴, —C(O)R¹⁴, and —S(O)₂R¹⁴; and each R¹⁴ is independently hydrogen, alkyl, alkanyl, alkynyl, aryl, arylalkyl, arylheteralkyl, arylaryl, heteroaryl or heteroarylalkyl.

“Prodrug” herein refers to a compound that is converted into the parent compound in vivo. Prodrugs often are useful because, in some situations, they may be easier to administer than the parent compound. For example, the prodrug may be bioavailable by oral administration while the parent compound is not. The prodrug may also have improved solubility in pharmaceutical compositions over the parent compound. An example of a prodrug would be a compound of the embodiments of the present invention that is administered, for example, as an ester (the “prodrug”) to facilitate transmittal across a cell membrane when water solubility is detrimental to mobility, but then is metabolically hydrolyzed to an active entity once inside the cell where water solubility is beneficial. Such a compound is generally inactive (or less active) until converted to the active form.

“Pharmaceutically acceptable salt” refers to a salt of a compound of the invention that is pharmaceutically acceptable and that possesses the desired pharmacological (e.g., anti-viral) activity. Such salts include the following: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-I-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine, and the like.

7-Deaza D-nucleoside Anti-viral Compounds and Derivatives Thereof

As set forth above, the present invention provides D-nucleoside analogues and derivatives thereof, pharmaceutically acceptable salts thereof, and uses thereof. In particular, the D-nucleoside analogues are 7-deaza-D-nucleoside analogues and derivatives thereof. By way of background, a number of strategies have been used in the attempt to treat chronic HBV infection, wherein a treatment can include roughly achieving the following three goals: (1) elimination of infectivity and transmission of HBV to others, 2) arresting the progression of liver disease and improving clinical prognosis, or 3) preventing development of cirrhosis and HCC. To date, a therapeutic agent that adequately treats or prevents an HBV infection and any associated disease has remained elusive. The instant invention provides certain 7-deaza-D-nucleoside analogues and derivatives thereof that have unexpectedly high anti-viral activity, and in particular high anti-viral activity against HBV.

In one preferred embodiment, the present invention provides compounds according to structure (I): 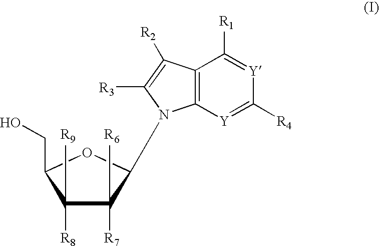
and pharmaceutically acceptable salts thereof wherein:
R¹ is hydrogen, C₁-C₆ alkyl, Cl, OH, C₁-C₄ alkoxy, NH₂, or NHZR⁵;
each of R² and R³ are independently hydrogen, C₁-C₆ alkyl, methyl, C₂-C₆ alkenyl, C₂-C₆ alkynyl, Cl, I, Br, F, heterocyclyl, or R² and R³ together with the carbons to which they are attached form a 5-membered ring;
R⁴ is hydrogen, OH, C₁-C₆ alkyl, C₁-C₆ alkenyl, C₁-C₄ alkoxy or NH₂, NHZR⁵, or N(R⁵)₂;
each R⁵ is independently C₁-C₆ alkyl, C₅-C₆ cycloalkyl, or aryl;
each of R⁶, R⁷, R⁸ and R⁹ are independently hydrogen, OH, C₁-C₆ alkyl, NH₂, NHZR⁵, F, Cl, or Br, or R⁶, R⁷, R⁸ and R⁹ form an epoxide or a double bond;
each of Y and Y′ are independently N or CH; and
Z is CO, C(O)NH, or SO₂.

As used herein, when referring to R⁶, R⁷, R⁸ and R⁹ forming an epoxide, typically in the context of the instant invention, this means the R⁶ and R⁹ will be bridged by an oxygen to form an epoxide, or the R⁷ and R⁸ will be bridged by an oxygen to form an epoxide. That is, an R⁶ and R⁸ epoxide, or an R⁷ and R⁹ epoxide, will typically not form. As used herein, when referring to R⁶, R⁷, R⁸ and R⁹ forming a double bond, this means, for example, that a double bond exists between the carbon attached to R⁶ and R⁷ and the carbon attached to R⁸ and R⁹ such that only one of R⁶ and R⁷ and simultaneously, only one of R⁸ or R⁹ remain, while the other groups have no substituent. Possible combinations that could have a substituent at the same time would be R⁶ and R⁸, or R⁶ and R⁹, or R⁷ and R⁸, or R⁷ and R⁹, with the other groups having no substituent.

In other preferred embodiments, anti-viral agents of the instant invention include compounds according to structure (I), and pharmaceutically acceptable salts thereof, wherein:
each R⁵ is independently C₁-C₆ alkyl, C₅-C₆ cycloalkyl, or aryl;
each of R⁶, R⁷, R⁸ and R⁹ are independently hydrogen, OH, C₁-C₆ alkyl, NH₂, NHZR⁵, F, Cl, or Br, or R⁶, R⁷, R⁸ and R⁹ form an epoxide or a double bond;
each of Y and Y′ are independently N or CH;
Z is CO, C(O)NH, or SO₂; and
(a) R¹ is NH₂; R² is a halogen or C₁-C₄ alkyl; and R³ and R⁴ are hydrogen; or
(b) R¹ is NH₂; R² is hydrogen or a halogen; R³ is a halogen or C₁-C₄ alkyl; and R⁴ is hydrogen; or
(c) R¹ is NH₂; each of R² and R³ are independently hydrogen or a halogen; and R⁴ is C₁-C₄ alkyl; or
(d) R¹ is NH₂; R² and R³ together with the carbon atoms they are attached to form a pentene ring, and R⁴ is hydrogen; or
(e) R¹ is hydrogen or C₁-C₄ alkyl; each of R² and R³ are independently hydrogen or a halogen; and R⁴ is hydrogen; or
(f) R¹ is NH₂; each of R² and R³ are independently hydrogen or a halogen; and R⁴ is NHZR⁵.

In further preferred embodiments, the present invention includes compounds according to structure (I), and pharmaceutically acceptable salts thereof, wherein:
R¹ is hydrogen, C₁-C₆ alkyl, Cl, OH, C₁-C₄ alkoxy, NH₂, or NHZR⁵;
each of R² and R³ are independently hydrogen, C₁-C₆ alkyl, methyl, C₂-C₆ alkenyl, C₂-C₆ alkynyl, Cl, I, Br, F, heterocyclyl, or R² and R³ together with the carbons to which they are attached form a 5-membered ring;
R⁴ is hydrogen, OH, C₁-C₆ alkyl, C₁-C₆ alkenyl, C₁-C₄ alkoxy or NH₂, NHZR⁵ or N(R⁵)₂;
each R⁵ is independently C₁-C₆ alkyl, C₅-C₆ cycloalkyl, or aryl;
each of Y and Y′ are independently N or CH;
Z is CO, C(O)NH, or SO₂; and
(a) R⁶, R⁷, R⁸ and R⁹ are hydrogen; or
(b) R⁶, R⁸ and R⁹ are hydrogen; and R⁷ is OH; or
(c) R⁶ and R⁹ are hydrogen; R⁷ is C₁-C₄ alkyl; and R⁸ is OH; or
(d) R⁶ and R⁹ are hydrogen; R⁷ is NHZR⁵; and R⁸ is OH; or
(e) R⁶ and R⁹ are hydrogen; R⁷ is F; and R⁸ is OH; or
(f) R⁶ is C₁-C₄ alkyl, R⁷ and R⁹ are hydrogen; and R⁸ is OH.

In a preferred embodiment, the present invention comprises a compound according to structure (II):

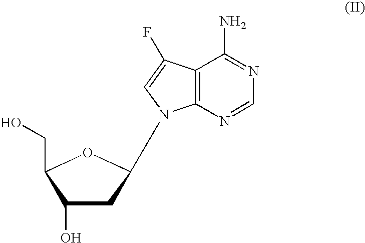

In still other embodiments, compounds of the invention include those compounds as described herein wherein the furanose ring is an open chain (rather than a closed ring), and wherein the bond between the oxygen and the 1′ carbon is omitted, the 1′ carbon is a methylene group, and the 4′ carbon is substituted, preferably with a hydroxyl group.

“Structurally pure” refers to a compound composition in which a substantial percentage, e.g., on the order of 95% to 100% and preferably ranging from about 95%, 96%, 97%, 98%, 99% or more, of the individual molecules comprising the composition each contain the same number and types of atoms attached to each other in the same order and with the same bonds. As used herein, “structurally pure” is not intended to distinguish different geometric isomers or different optical isomers from one another. For example, as used herein a mixture of cis- and trans-but-2,3-ene is considered structurally pure, as is a racemic mixture. When compositions are intended to include a substantial percentage of a single geometric isomer or optical isomer, the nomenclature “geometrically pure” and “optically or enantiomerically pure,” respectively, are used.

The phrase “structurally pure” is also not intended to discriminate between different tautomeric forms or ionization states of a molecule, or other forms of a molecule that result from equilibrium phenomena or other reversible interconversions. Thus, a composition of, for example, an organic acid is structurally pure even though some of the carboxyl groups may be in a protonated state (COOH) and others may be in a deprotonated state (COO⁻). Likewise, a composition comprising a mixture of keto and enol tantomers, unless specifically noted otherwise, is considered structurally pure.

Methods of Synthesis

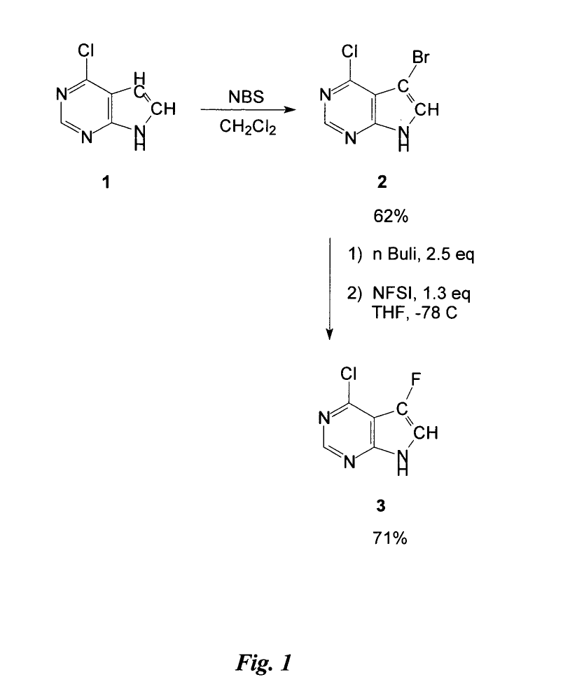

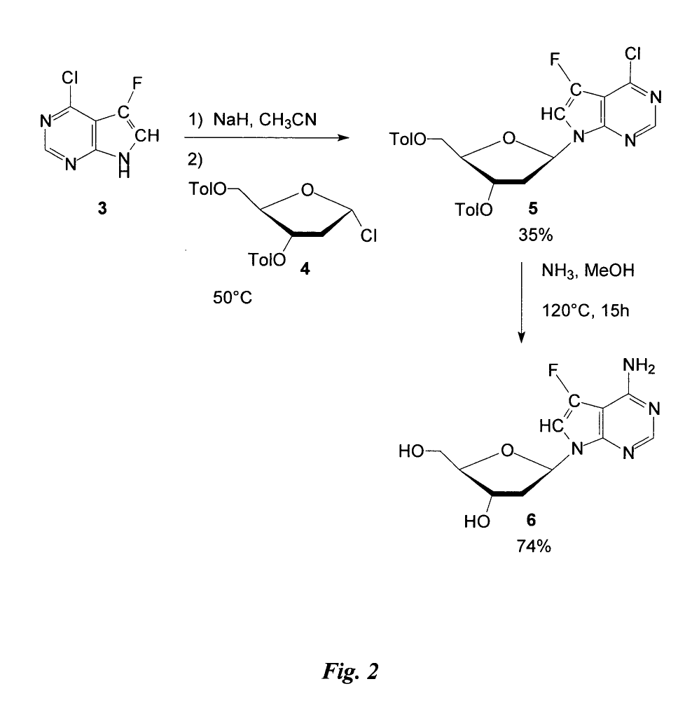

The compounds of the invention can be synthesized via several different synthetic routes using commercially available starting materials or starting materials prepared by conventional synthetic or biosynthetic methods. A general synthetic approach of an exemplary compound of the invention is illustrated in FIGS. 1 and 2.

The following exemplary reaction is merely for illustrative purposes and a person having ordinary skill in the art would understand that different reactants can be used to generate the compounds of the instant invention. Briefly, 6-chloro-7-deazapurine 1 can be used as a starting compound, which is first brominated with N-bromosuccinimide (NBS) in dichloromethane to give 4-chloro-5-bromo-7 H-pyrrolo[2, 3-d] pyrimidine 2 (see Townsend, J. Med. Chem. 31:2086, 1988). This reaction is followed with a halogen metal exchange using, for example, n-butyllithium (nBuLi), before quenching with N-fluorobenzene sulfonimide (NFSI) to provide 4-chloro-5-fluoro-7 H-pyrrolo[2, 3-d] pyrimidine 3. Other electrophilic fluorination reagents, such as 1-chloromethyl-4-fluoro-1,4-diazoniabicyclo[2.2.2]octane bis(tetrafluroborate) (F-TEDA-BF-4), N-fluoropyridinium triflate, N-fluoroquinuclidinium triflate, and the like, could also be used in this process. The coupling reaction between 4-chloro-5-fluoro-7 H-pyrrolo[2,3-d] pyrimidine 3 salt (obtained by adding, for example, sodium hydride in acetonitrile) and 2′deoxy-3′, 5′di-O-p-toluoyl-α-D-erythro-pentofuranosyl chloride 4 (see, e.g., Hoffer, M. Chem. Ber. 93: 2777, 1960) will yield 4-chloro-5-fluoro-7-(2′-deoxy-3′, 5′-di-O-p-toluoyl-β-D-erythro-pentofuranosyl) pyrrolo[2,3-d] pyrimidine 5. The treatment of compound 5 with methanolic ammonia allows the conversion of chlorine to an amine with a subsequent removal of the toluoyl groups to provide a compound of the instant invention, 4-amino-5-fluoro-7-(2′-deoxy-β-D-erythro-pentofuranosyl) pyrrolo[2,3-d]pyrimidine 6.

Therapeutic Formulations and Methods of Use

As described herein, compounds of the instant invention show surprising and exceptionally strong inhibition of viral replication, particularly HBV. Compounds of the invention, including the compound of structure (II), exhibit anti-viral activity against HBV. Certain compounds exhibit anti-viral activity against HBV with an EC₅₀ below 2 μM in an HBV cell-based assay. In certain embodiments, the invention provides compounds capable of inhibiting viral replication, preferably HBV, at clinically relevant concentrations.

HBV cell-based assays for the evaluation of anti-viral activity against HBV are known in the art (see, e.g., Korba et al., Antiviral Res. 15:217, 1991; and Korba et al., Antiviral Res. 19:55, 1992). Moreover, the compounds of the invention can be useful research tools for in vitro and cell-based assays to study the biological mechanisms of viral infection, growth, and replication, preferably for HBV. By way of background and not wishing to be bound by theory, HBV replication is unusual in that this virus has a partially single-stranded circular DNA (all of the minus DNA strand and part of the plus strand), but replicates by way of an RNA intermediate (typically, single-stranded viral DNA is converted to double stranded DNA, which serves as the template for viral replication). That is, when the virus enters the cell, the minus-strand DNA is transported to the cell nucleus, plus strand RNA is then transcribed from the DNA, the RNA is transported to the cytoplasm, and reverse transcriptase then synthesizes viral DNA from the RNA for packaging. In one preferred embodiment, the invention provides a method of identifying anti-viral compounds, comprising contacting a host cell infected with a virus with a candidate 7-deaza-D-nucleoside compound or derivative thereof of the invention for a time sufficient to inhibit viral replication, and identifying a candidate compound that inhibits viral replication. In another embodiment, there is provided a method of identifying cells suspected of having a viral infection, comprising contacting a host cell suspected of being infected with a virus with a candidate 7-deaza-D-nucleoside compound or derivative thereof of the invention for a time sufficient to inhibit viral replication, and identifying cells infected with a virus. Preferably, the viral infection is caused by or associated with HBV.

In addition, in vivo models, such as woodchucks and Peking duck, for evaluating compounds for anti-viral activity against HBV are known in the art (see, e.g., Tennant et al., ILAR Journal 42:89, 2001; Zuckerman, J. Virology Methods, 17:119, 1987; Aguesse-Germon et. al., Antimicrobial Agents and Chemotherapy 42:369, 1998). Furthermore, as a person having ordinary skill in the art would understand, these in vitro and in vivo assays can be used to determine the therapeutic value of a candidate compound and what dosage parameters would be most useful in treating a subject in need thereof for a viral infection, preferably the subject is a mammal, and most preferably is human.

The invention also relates to pharmaceutical compositions that contain one or more compounds used to treat or prevent a viral infection (e.g., HBV). The invention further relates to methods for treating or preventing viral infections by administering to a subject a 7-deaza-D-nucleoside compound or derivative thereof of the invention, or a mixture of such compounds at a dose sufficient to treat or prevent a viral infection, as described herein. The 7-deaza-D-nucleoside compounds and derivatives thereof, or cocktail of such compounds, are preferably part of a pharmaceutical composition when used in the methods of the present invention.

In preferred embodiments of the invention, the 7-deaza-D-nucleoside compounds or derivatives thereof described herein are used to treat or prevent a viral infection in a subject, preferably the subject is a mammal, even more preferably a human. In other preferred embodiments, the viral infection is due to HBV or other single-stranded DNA viruses. Certain compounds of the instant invention, including the compound of the structure (II), show good overall biopharmaceutical properties and are orally available. In one embodiment, the invention comprises a pharmaceutical composition comprising a 7-deaza-D-nucleoside anti-viral compound as described herein (or a pharmaceutically active derivative thereof and a pharmaceutically acceptable carrier, excipient or diluent. Preferably, the pharmaceutical composition comprises an anti-viral compound that has structure (II). The term “pharmaceutically active derivative” refers to any compound that, upon administration to a subject in need thereof, is capable of providing directly or indirectly (e.g., a pro-drug) the compounds of the instant invention.

As set forth herein, the active compound may be included in a pharmaceutically acceptable carrier or diluent for administration to a subject in need thereof in an amount effective to treat or prevent an HBV infection. A preferred dose of the active compound for all of the above-mentioned indications is in a range from about 0.01 mg/kg to about 300 mg/kg per day; preferably about 0.1 mg/kg to about 100 mg/kg per day, more preferably about 0.5 mg/kg to about 25 mg/kg body weight of the recipient per day. A typical topical dosage will range from about 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to a person having ordinary skill in the art.

The methods of the invention comprise administration to a mammal (preferably human) suffering from a variety of forms of cancer, arthritis, and diseases related to angiogenesis in which ADAM-10 (an acronym for “a disintegrin and metalloproteinase”-10) plays a critical role. In one embodiment, there is provided a pharmaceutical composition according to the invention in an amount sufficient to alleviate the condition of interest. The compound can be conveniently administered in any suitable unit dosage form, including one containing from about 1 mg to about 3000 mg, and preferably about 5 mg to about 500 mg of active ingredient per unit dosage. In one preferred embodiment, an oral dosage of about 1 mg to about 500 mg, preferably about 10 mg to about 250 mg, and more preferably about 25 mg to about 250 mg is administered to a subject to treat or prevent a viral infection.

The active ingredient should be administered to achieve peak plasma concentrations of the active compound of about 0.001 μM to about 30 μM, and preferably about 0.01 μM to about 10 μM. This may be achieved, for example, by intravenous injection of a composition or formulation of a 7-deaza-D-nucleoside compound or derivative thereof of the invention, optionally in saline or other aqueous medium. In another embodiment, a 7-deaza-D-nucleoside compound or derivative thereof of the invention or composition thereof is administered as a bolus.

The concentration of active compound in a pharmaceutical composition of the instant invention will depend on absorption, distribution, inactivation, and excretion rates of the drug, as well as other factors known to those of skill in the art. It is to be understood that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid, Primogel, or corn starch; a lubricant such as magnesium stearate or Sterores; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material of the above type, a liquid carrier such as a fatty oil. In addition, dosage unit forms can contain various other materials which modify the physical form of the dosage unit, for example, coatings of sugar, shellac, or enteric agents. See generally “Remington's Pharmaceutical Sciences,” Mack Publishing Co., Easton, Pa.

The active compound or pharmaceutically acceptable salt or derivative thereof can be administered as a component of an elixir, suspension, syrup, wafer, chewing gum or the like. A syrup may contain, in addition to the active compounds, sucrose as a sweetening agent and certain preservatives, dyes and colorings and flavors.

The pharmaceutical composition of the instant invention will preferably include at least one of a pharmaceutically acceptable vehicle, carrier, diluent, or excipient, in addition to one or more 7-deaza-D-nucleoside compound or derivative thereof of the invention and, optionally, other components. A composition of the invention may have a variety of active ingredients, such as a 7-deaza-D-nucleoside compound or derivative thereof, or a cocktail of two or more 7-deaza-D-nucleoside compounds or derivatives thereof, or a cocktail of one or more 7-deaza-D-nucleoside compounds or derivatives thereof with one or more antibiotic, antifungal, anti-inflammatory, or other anti-viral compound. Pharmaceutically acceptable carriers suitable for use with a composition may include, for example, a thickening agent, a buffering agent, a solvent, a humectant, a preservative, a chelating agent, an adjuvant, and the like, and combinations thereof. Pharmaceutically acceptable carriers for therapeutic use are well known in the pharmaceutical art, and as described herein and, for example, in Remington's Pharmaceutical Sciences, Mack Publishing Co. (A. R. Gennaro, ed., 18^{th} Edition, 1990) and in CRC Handbook of Food, Drug, and Cosmetic Excipients, CRC Press LLC (S. C. Smolinski, ed., 1992).

Solutions or suspensions used for parenteral, intradermal, subcutaneous, or topical application can include the following components: a sterile diluent such as water for injection, saline solution, fixed oils, polyethylene glycols, glycerin, propylene glycol or other synthetic solvents; anti-bacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parental preparation can be enclosed in ampoules, disposable syringes or multiple dose vials made of glass or plastic. If administered intravenously, preferred carriers are physiological saline or phosphate buffered saline (PBS) or an adjuvant. Exemplary adjuvants are alum (aluminum hydroxide, REHYDRAGEL®), aluminum phosphate, virosomes, liposomes with and without Lipid A, Detox (Ribi/Corixa), MF59, or other oil and water emulsions type adjuvants, such as nanoemulsions (see, e.g., U.S. Pat. No. 5,716,637) and submicron emulsions (see, e.g., U.S. Pat. No. 5,961,970), and Freund's complete and incomplete. In one preferred embodiment, a pharmaceutical composition of the invention is sterile.

In certain embodiments, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. For example, as is known in the art, some of these materials can be obtained commercially from Alza Corporation (CA) and Gilford Pharmaceuticals (Baltimore, Md.).

Liposomal suspensions may also be pharmaceutically acceptable carriers. These may be prepared according to methods known to those skilled in the art, for example, as described in U.S. Pat. No. 4,522,811. For example, liposome formulations may be prepared by dissolving appropriate lipid(s) (such as stearoyl phosphatidyl ethanolamine, stearoyl phosphatidyicholine, arachadoyl phosphatidylcholine, and cholesterol) in an inorganic solvent that is then evaporated, leaving behind a thin film of dried lipid on the surface of the container. An aqueous solution of the active compound or its monophosphate, diphosphate, and/or triphosphate derivatives are then introduced into the container. The container is then swirled by hand to free lipid material from the sides of the container and to disperse lipid aggregates, thereby forming the liposomal suspension.

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. The invention having been described, the following examples are intended to illustrate, and not limit, the invention.

### EXAMPLES

In the examples below, the following abbreviations have the following meanings. Any abbreviations not defined have their generally accepted meaning. Unless otherwise stated, all temperatures are in degrees Celsius.

| | |
|---|---|
| DMSO = | dimethyl sulfoxide |
| EtOAc = | ethyl acetate |
| TFA = | trifluoroacetic acid |
| THF = | tetrahydrofuran |
| MeOH = | methanol |
| DCC = | 1,3-dicyclohexylcarbodiimide |
| DMAP = | 4-(dimethylamino)pyridine |
| DMF = | dimethyl formamide |
| DIEA = | N,N-diisopropylethylamine |
| NaOMe = | sodium methoxide |

General: Unless noted otherwise, reagents, starting material and solvents were purchased from commercial suppliers (Aldrich, Fluka, Sigma, and etc.), and used without further purification; reactions were run under nitrogen atmosphere; reaction mixtures were monitored by thin layer chromatography (silica TLC), analytical high performance liquid chromatography (anal. HPLC), or mass spectrometry; reaction mixtures were commonly purified by flash column chromatography on silica gel, or by preparative HPLC using the general protocol described below; NMR samples were dissolved in deuterated solvent (CD₃OD, CDCl₃, or DMSO-d6), and spectra were acquired with a Varian Gemini 2000 instrument (500 MHz) under standard parameters; and mass spectrometric identification was performed by an electrospray ionization method (ES-MS) with a Waters 2690 Alliance System.

### Example 1

### Preparation of Intermediate 4-Chloro-5-Fluoro-7H-Pyrrolo[2,3-d] Pyrimidine

4-Chloro-5-bromo-7H-pyrrolo[2,3-d] pyrimidine 2 (0.6 g, 2.60 mmol) was dissolved in 30 mL THF (dry), then cooled to −78° C. before adding 4.10 mL nBuLi (1.6 M in hexane) dropwise over a period of 10 minutes. The mixture was stirred for 20 minutes at −78° C. before adding 3.38 mmol N-fluorobenzene sulfonimide (NFSI) (1.065 g in 6.5 mL dry THF) dropwise over a period of 15 minutes. The mixture was warmed to room temperature overnight, quenched with 2 mL water, and then evaporated to dryness. The crude preparation obtained was partitioned between ethyl acetate (EtOAc) and a saturated solution of ammonium chloride (40 mL/20 mL) then the aqueous layer was extracted with 20 mL of EtOAc and the combined organic layers were washed with 20 mL water. The organic layer was dried with MgSO₄, filtered, and the solvent was evaporated. The crude was purified on silica-gel (silica-gel solid deposit in MeOH) with 4% MeOH/CH₂Cl₂ to provide the title compound (0.36 g, 71% yield); ¹H NMR (DMSO):δ 7.70 (s, 1H); 8.62 (s, 1H); 12.25 (s, NH).

### Example 2

### Preparation of Intermediate 4-Chloro-5-Fluoro-7-(2′-Deoxy-3′, 5′-di-O-p-Toluoyl-β-D-Erythro-Pentofuranosyl) Pyrrolo[2,3] Pyrimidine

Sodium hydride 95% (0.032 g, 1.25 mmol in acetonitrile) was added to 4-chloro-5-fluoro-7H-pyrrolo[2,3-d] pyrimidine 3 (0.21 g, 1.22 mmol). The mixture was stirred for 30 minutes at room temperature and then 2′deoxy-3′, 5′di-O-ρ-toluoyl-α-D-erythro-pentofuranosyl chloride 4 (0.5 g, 1.28 mmol) was added over a period of 10 minutes. This mixture was then stirred for 2 hours at 50° C., filtered, and the solvent evaporated. The crude preparation was purified on silica-gel (silica-gel solid deposit in EtOAc) with a gradient of EtOAc/hexane (8 to 15%) to provide the title compound (0.322 g, 50% yield); ¹H NMR (DMSO): δ 2.36 (s, 3H); 2.39 (s, 3H); 2.66-2.8 (m, 1H); 3.05-3.18 (m, 1H); 4.45-4.65 (m, 3H); 5.65-5.68 (m, 1H); 6.79 (t, 1H, J=7.31 Hz); 7.29 (d, 2H, J=8.29 Hz); 7.36 (d, 2H, J=8.29 Hz); 7.83 (d, 2H, J =8.29 Hz); 7.94 (d, 2H, J=8.29 Hz); 8.01 (d, 1H, J=1.95 Hz); 8.68 (s, 1H).

### Example 3

### 4-Amino-5-Fluoro-7-(2′-Deoxy-β-D-Erythro-Pentofuranosyl) Pyrrolo[2,3-d] Pyrimidine

4-Chloro-5-fluoro-7-(2′-deoxy-3′,5′-di-O-p-toluoyl-β-D-erythro-pentofuranosyl) pyrrolo[2,3-d] pyrimidine 5 (0.05 g, 0.095 mmol) was dissolved in 15 mL dry MeOH in a sealed tube. The mixture was saturated with ammonia gas at 0° C. and then heated at 126° C. for 15 hours. Solvent was evaporated before the crude preparation was partitioned between ether and water (30 mL/15 mL). The pH of the aqueous layer was adjusted to 7 with 3N HCl and then the water was evaporated before purification on a C-18 column with water to provide the title compound (0.019 mg, 74% yield); ¹H NMR (DMSO): δ 2.08-2.18 (m, 1H); 2.27-2.43 (m, 1H); 3.40-3.58 (m, 2H); 4.22-4.35 (m, 1H); 4.99 (t, 1H, J=5.8 Hz); 5.22 (d, 1H, J=3.91 Hz); 6.52 (t, 1H, J=6.84 Hz); 6.96 (s, NH2); 7.30 (d, 1H, J=1.95 Hz); 8.04 (s, 1H).

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. An anti-viral compound and pharmaceutically acceptable salts thereof, according to structural formula (I): 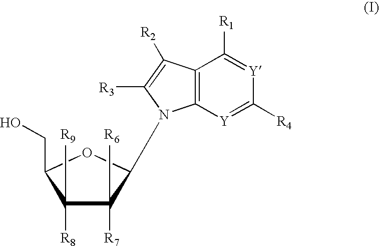
wherein:
R¹ is hydrogen, C₁-C₆ alkyl, Cl, OH, C₁-C₄ alkoxy, NH₂, or NHZR⁵;
each of R² and R³ are independently hydrogen, C₁-C₆ alkyl, methyl, C₂-C₆ alkenyl, C₂-C₆ alkynyl, Cl, I, Br, F, heterocyclyl, or R² and R³ together with the carbons to which they are attached form a 5-membered ring;
R⁴ is hydrogen, OH, C₁-C₆ alkyl, C₁-C₆ alkenyl, C₁-C₄ alkoxy, NH₂, NHZR⁵ or N(R⁵)₂;
each R⁵ is independently C₁-C₆ alkyl, C₅-C₆ cycloalkyl, or aryl;
each of R⁶, R⁷, R⁸ and R⁹ are independently hydrogen, OH, C₁-C₆ alkyl, NH₂, NHZR⁵, F, Cl, or Br, or R⁶, R⁷, R⁸ and R⁹ form an epoxide or a double bond;
each of Y and Y′ are independently N or CH; and
Z is CO, C(O)NH or SO₂.

2. The compound according to claim 1 wherein R¹ is NH₂; R² is a halogen or C₁-C₄ alkyl; and R³ and R⁴ are hydrogen.

3. The compound according to claim 1 wherein R¹ is NH₂; R² is hydrogen or a halogen; R³ is a halogen or C₁-C₄ alkyl; and R⁴ is hydrogen.

4. The compound according to claim 1 wherein R¹ is NH₂; each of R² and R³ are independently hydrogen or a halogen; and R⁴ is C₁-C₄ alkyl.

5. The compound according to claim 1 wherein R¹ is NH₂; R² and R³ together with the carbon atoms they are attached to form a pentene ring; and R⁴is hydrogen.

6. The compound according to claim 1 wherein R¹ is hydrogen or C₁-C₄ alkyl; each of R² and R³ are independently hydrogen or a halogen; and R⁴ is hydrogen.

7. The compound according to claim 1 wherein R¹ is NH2; each of R² and R³ are independently hydrogen or a halogen; and R⁴ is NHZR⁵; wherein Z and R⁵ are as defined in claim 1.

8. The compound according to claim 1 wherein R⁶, R⁷, R⁸ and R⁹ are hydrogen.

9. The compound according to claim 1 wherein R⁶, R⁸ and R⁹ are hydrogen; and R⁷ is OH.

10. The compound according to claim 1 wherein R⁶ and R⁹ are hydrogen; R⁷ is C₁-C₄ alkyl; and R⁸ is OH.

11. The compound according to claim 1 wherein R⁶ and R⁹ are hydrogen; R⁷ is NHZR⁵; and R⁸ is OH; wherein Z and R⁵ are as defined in claim 1.

12. The compound according to claim 1 wherein R⁶ and R⁹ are hydrogen; R⁷ is F; and R⁸ is OH.

13. The compound according to claim 1 wherein R⁶ is C₁-C₄ alkyl, R⁷ and R⁹ are hydrogen; and R⁸ is OH.

14. The compound according to claim 1 wherein said compound has structure (II):

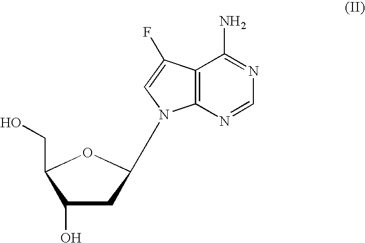

15. A pharmaceutical composition comprising a compound according to any one of claims 1 to 14 and a pharmaceutically acceptable carrier, excipient or diluent.

16. A method of treating or preventing a viral infection, comprising administering to a subject in need thereof an anti-viral compound according to any one of claims 1 to 14 in an amount effective to treat or prevent a viral infection.

17. The method of claim 16 in which said anti-viral compound is administered orally.

18. The method of claim 16 wherein said anti-viral compound is administered systemically.

19. The method of claim 16 wherein said viral infection is caused by hepatitis B virus (HBV).

20. A method of treating or preventing a viral infection, comprising administering to a subject in need thereof a composition according to claim 15 in an amount effective to treat or prevent a viral infection.
